SEQUENCE LISTING (10 sequences)

SEQ ID NO: 1 - DNA, 339 bp, Homo sapiens
  CDS (1)..(339)
ctcgaggagtctggagcagaagtgaagaagccgggcgaaaatcttaagatctcctgcgaggcttctggatacaattttgtcaatcactggatcggctgggtgcgccagatgcccgggagaggccttgagtggatgggccgcatctatcctggagactctgaaaccagattcagtccgtccttccaagggcaggtcaccatctcagtcgacaaaactctgagtaccgcctccctacagtggaacagtctcaagacgtcggacagcgccacatattattgtgtgacactggggcgctggagcagctggcaaggtggggcgctctcatggggccagggaacc

SEQ ID NO: 2 - protein, 113 aa, Homo sapiens
LEESGAEVKKPGENLKISCEASGYNFVNHWIGWVRQMPGRGLEWMGRIYPGDSETRFSPSFQGQVTISVDKTLSTASLQWNSLKTSDSATYYCVTLGRWSSWQGGALSWGQGT

SEQ ID NO: 3 - DNA, 303 bp, Homo sapiens
  CDS (1)..(303)
gagctcacgcagtctccagccaccgtgtctgtgtctccaggggaaagagccaccctctcctgcagggccagtcagagtattagtttccacttagcctggtaccagcagaaacctggccaggctcccagtctcctcatctacggaacatccaccagggccactggtatcccagccaggttcagtggcagtgggtctgggacagagttcactctcaccatcagcagcctgcagtctgaagattctgcggtttattactgtcagcagtatcataactggcctcccctcactttcggcggagggacc

SEQ ID NO: 4 - protein, 101 aa, Homo sapiens
ELTQSPATVSVSPGERATLSCRASQSISFHLAWYQQKPGQAPSLLIYGTSTRATGIPARFSGSGSGTEFTLTISSLQSEDSAVYYCQQYHNWPPLTFGGGT

SEQ ID NO: 5 - protein, 12 aa, Artificial sequence
  synthetic peptide
TMGFTAPRFPHY

SEQ ID NO: 6 - protein, 12 aa, Artificial sequence
  synthetic peptide
MQSPFTPHFAER

SEQ ID NO: 7 - protein, 12 aa, artificial sequence
  synthetic peptide
MQSPIVLPLSLS

SEQ ID NO: 8 - protein, 12 aa, artificial sequence
  synthetic peptide
HHEPGAWLPLSP

SEQ ID NO: 9 - protein, 17 aa, homo sapiens
CVTLGRWSSWQGGALSW

SEQ ID NO: 10 - protein, 12 aa, homo sapiens [sequence omitted]

(19) United States
(12) Patent Application Publication
(10) Pub. No.: US 2012/0115741 A1
(43) Pub. Date: May 10, 2012

(54) Reagents For The Atherosclerotic Coronary Plaque And Uses Thereof

(75) Inventors: Massimo Clementi, Milano (IT); Roberto Burioni, Milano (IT)

(73) Assignee: BRACCO IMAGING S.P.A., Milano (IT)

(21) Appl. No.: 13/203,060

(22) PCT Filed: Mar. 17, 2010
(86) PCT No.: PCT/EP2010/053430

§ 371 Date: Aug. 24, 2011

(30) Foreign Application Priority Data
Mar. 19, 2009 (EP) 09155614.2

(51) Int. Cl. C40B 30/04; C12Q 1/70; C12P 21/02; C07K 16/18; G01N 33/566

(52) U.S. Cl. 506/9; 530/389.1; 435/7.92; 435/69.6; 435/7.1; 435/5

(57) ABSTRACT

The present invention discloses antibodies or fragments thereof able to bind isolated coronary plaque samples and processes for their production using host cells containing DNA sequences encoding for said antibodies of fragments thereof. Methods for screening ligands to said isolated samples are also described, and compositions containing said antibodies are also provided.

The present invention relates to reagents and a method for preparing selves. In particular, the present invention encompasses antibodies or fragments thereof that are directed against antigens present in the coronary plaque. The present invention further relates to the nucleotidic sequences coding for these antibodies and amino acidic sequences of the antibodies or fragments thereof for use in immunoassays. Further, the invention encompasses diagnostic and therapeutic applications related to the use of said antibodies or fragments thereof or of their ligands.

### BACKGROUND ART

The acute coronary syndrome (also shortly referred to as ACS) is the manifestation of a plaque rupture in a coronary artery.

The rupture or the erosion of an atherosclerotic plaque, with the subsequent formation of thrombus and occlusion of the artery may cause myocardial infarction and unstable angina (see, for a general reference, “New insights into atherosclerotic plaque rupture” D. M. Braganza and M. R. Bennett, Postgrad. Med. J. 2001; 77; 94-98).

An atherosclerotic event begins as a subendothelial accumulation of lipid laden, monocytes derived foam cells and associated T cells which form a non-stenotic fatty streak. With progression, the lesion takes the form of an acellular core of cholesterol esters, bounded by an endothelialised fibrous cap containing smooth muscle cells (VMSC) and inflammatory cells (both macrophages and T lymphocytes). Also presented in the advanced lesions are new blood vessels and deposits of calcium hydroxyapatite may also appear in advanced lesions (see as a general reference, “Coronary disease: Atherogenesis: current understanding of the causes of atheroma” Peter L. Weissberg, Heart 2000; 83; 247-252).

The extracellular lipid core of the plaque is composed of free cholesterol, cholesterol crystals and cholesterol esters derived from lipids infiltrated the arterial wall or derived from the dead foam cells. The lipid core may affect the plaque by causing stress to the shoulder regions of the plaque; in addition, the lipid core contains prothrombotic oxidized lipids and it is further impregnated with tissue factors derived from macrophages in which the lipid core materials are highly thrombogenic when exposed to circulating blood (see, for instance, “Mechanism of Plaque Vulnerability and Rupture” Prediman K. Shah, Journal of the American College of Cardiology 2003; 41(Suppl 1); 15-22).

The stability of the plaque depends also upon the vascular smooth muscle cells (SMCs) content of the plaque, as they are capable of synthesising the structurally important collagens types I and III. In contrast, macrophages and others inflammatory cells may release matrix metalloproteinases (MMPs) which degrade collagen and extracellular matrix, thus potentially weakening the plaque (see, “New insights into atherosclerotic plaque rupture” D. M. Braganza and M. R. Bennett, Postgrad. Med. J. 2001; 77; 94-98).

The structural components of the fibrous cap include matrix component such as collagen, elastin and proteoglycans, derived from SMCs. Said fibrous cap protects the deeper components of the plaque from contact with circulating blood and has been observed to thin out in the vicinity of the rupture.

Ruptured plaques have been shown to have several histomorphologic features with respect to intact plaques. Therefore, when they are present, they are thought to indicate vulnerability to plaque rupture.

One of possible causes inherent to the plaque formation is thought to be caused by repeated injury to endothelium caused by the four “major” risk factors: smoking, hypertension, diabetes and hyperlipidaemia (high level of LDL and low level of HDL). Endothelial dysfunction following injury, moreover, plays a crucial role in plaque initiation, as lipids may pass more easily from the bloodstream into the tunica intima.

The rupture of a vulnerable plaque may occur either spontaneously, i.e. without occurrence of any of the above mentioned triggers or following a particular event, such as an extreme physical activity, a severe emotional trauma and stresses of different nature or acute infection.

Plaque rupture often leads to thrombosis with clinical manifestations of an ACS. The thrombotic response to a plaque rupture is probably regulated by the thrombogenicity of the constituents exposed on the plaque; generally, the plaque rupture develops in a lesion with a necrotic core and an overlying thin fibrous cap heavily infiltrated by inflammatory cells. A luminal thrombus further develops due to the physical contact between platelets and the necrotic core (see, for example, “Pathologic assessment of the vulnerable human coronary plaque” F. D. Kolodgie et al. Heart 2004; 90; 1385-1391).

Rupture or erosion of the fibrous cap exposes the highly thrombogenic collagenous matrix and lipid core to circulation leading inevitably to platelet accumulation and activation. This in turn leads to fibrin deposition and thrombus formation which may result into vessel occlusion, the latter being not inevitable, such as in the case of silent episodes (see, for instance, “Coronary disease: Atherogenesis: current understanding of the causes of atheroma” Peter L. Weissberg, Heart 2000; 83; 247-252).

Until recently, atherosclerosis was thought of as a degenerative and slowly progressive disease causing symptoms through its mechanical effects on blood flow, while it is now understood to be a dynamic inflammatory process that is eminently modifiable. Recent researches on cellular and molecular events underlying development and progression of atherosclerosis, focus the attention on the dynamic interaction between the plaque components that dictates the outcome of the disease (see, as a general reference “Coronary disease: Atherogenesis: current understanding of the causes of atheroma” Peter L. Weissberg, Heart 2000; 83; 247-252).

There are contrasting data for a relation between coronary syndrome and several pathogens to be assessed.

In a prospective study (see, for example, “Impact of viral and bacterial infectious burden on long term prognosis in patients with coronary artery disease” Rupprecht H. J. et al., Circulation 2001, Jul. 3; 104(1): 25-31) it was described the relation between stroke and 8 different pathogens (Herpes simplex virus 1-2, Epstein-Barr, Cytomegalovirus, Haemophilus influenzae, Mycoplasma pneumoniae, Helicobacter pylori and Chlamydia pneumoniae) in a group of 1018 patients; there was found an increase in mortality, related to the serum positivity for six to eight pathogens of 7% and 12.6% respectively.

De Palma and his group (“Patients with Acute Coronary Syndrome Show Oligoclonal T-Cell Recruitment Within Unstable Plaque” De Palma et al. Circulation 2006, 113: 640-646) conducted a study on the T cells repertoire recovered from blood sample and also directly from the coronary plaque of patients with acute coronary syndrome.

### Antibody Structures

There exist five types of antibodies (also called immunoglobulins): IgG, IgA, IgD, IgM and IgE. The structure of IgG, depicted in FIG. 1, comprises two light chains of a molecular weight of approximately 23 KDa and two heavy chains of about 53-70 KDa. The four chains being linked to each other by disulfide bonds in a “Y” configuration.

Heavy chains are classified as γ, η, α, δ and ε with some subclasses among them, while light chains are classified as either κ or λ.

Each heavy chain comprises a constant region and a variable region, the latter being located at the N-terminal end of the immunoglobulin molecule of approximately 100 amino acids in length.

In particular, the most variable part of the immunoglobulin (Ig) heavy and light chains is the third complementarity-determining region (CDR3), a short amino acid sequence which is formed by the junctions between the V-D-J gene segments. CDR3 is found in the variable domains of antigen receptor (e.g. immunoglobulin and T cell receptor) protein that complements an antigen.

The variability of the CDR3 portion is responsible of the elevated number of antibodies produced and which are specific for any antigens; said variability is determined by the rearrangement of the V, D and J minigenes that occurs in the bone marrow during the generation of mature B cells.

After this first rearrangement has occurred, when the mature B cell encounters an antigen, further hypermutational events are responsible for the increased affinity of the antibody for that specific antigen.

### SUMMARY OF THE INVENTION

The present invention relates to a recombinant antibody comprising amino acid SEQ ID NO: 2 and 4 or any fragment thereof, as reagents useful for the identification of antigens involved or present in the atherosclerotic plaque. Said antibody is preferably a IgG antibody.

It's a further object of the present invention a process for the production of said antibody, said method comprising the preparation of an expression system for the isolated polynucleotidic sequences encoding SEQ ID NO: 2 and 4 or any fragment thereof, in a host cell.

Preferably said isolated polynucleotidic sequences encoding SEQ ID NO: 2 and 4 are the polynucleotidic molecules SEQ ID NO:1 and 3 or fragments thereof.

Further embodiments encompass the expression vector, comprising one or more of the isolated polynucleotidic molecules of SEQ ID NO: 1 and 3, as well as the complementary or homologous sequences thereof in a suitable host cell, altogether representing an expression system.

It is a further object of the present invention an assay including the use of one or more of the amino acidic sequences corresponding to SEQ ID NO: 2 and 4 or any fragment thereof and the homologous sequences thereof.

In a further embodiment of the invention, there is provided a composition comprising the antibodies of the invention or any fragments thereof and a therapeutic or a diagnostic moiety linked thereto for therapeutic or diagnostic purposes.

Further object of the invention is a method for screening and/or identifying molecules or antigens, even comprised within a complex matrix, such as a cell or a biopsy specimen, having a binding affinity for the antibodies of the present invention or any fragments thereof.

### BRIEF DESCRIPTION OF THE DRAWINGS

FIG. 1 is a schematic representation of the structure of an IgG antibody molecule and of a Fab fragment thereof.

FIG. 2 represents the recombinant pattern for the production of antibodies.

FIG. 3 represents the number of functional gene segments in human immunoglobulin loci.

FIG. 4 is a schematic representation of the preparation of the antibodies or fragments thereof according to the present invention (left, mRNA from coronary plaque specimen) and according to the prior art (right, mRNA from blood samples).

FIG. 5 shows the results of immunoaffinity selection by Biopanning on cell lysate (Hep-2), carotid lysate, artificial antigens.

FIG. 6 shows the ELISA results for Fab 68 according to the present invention.

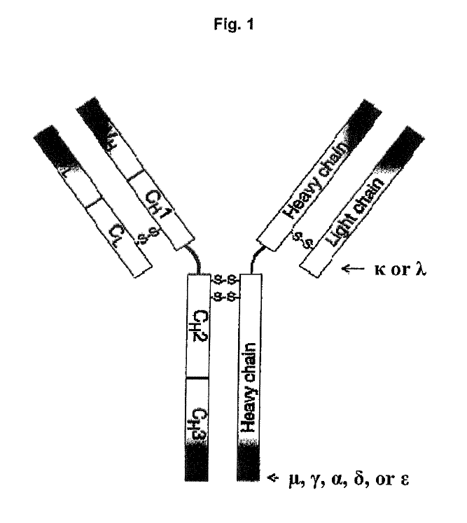

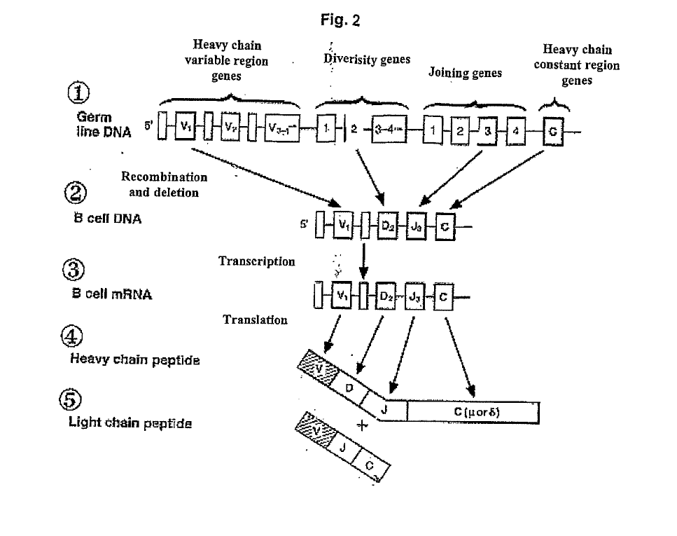

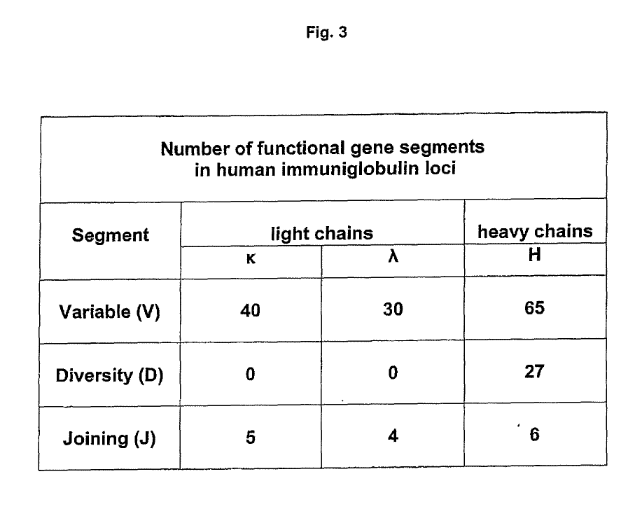

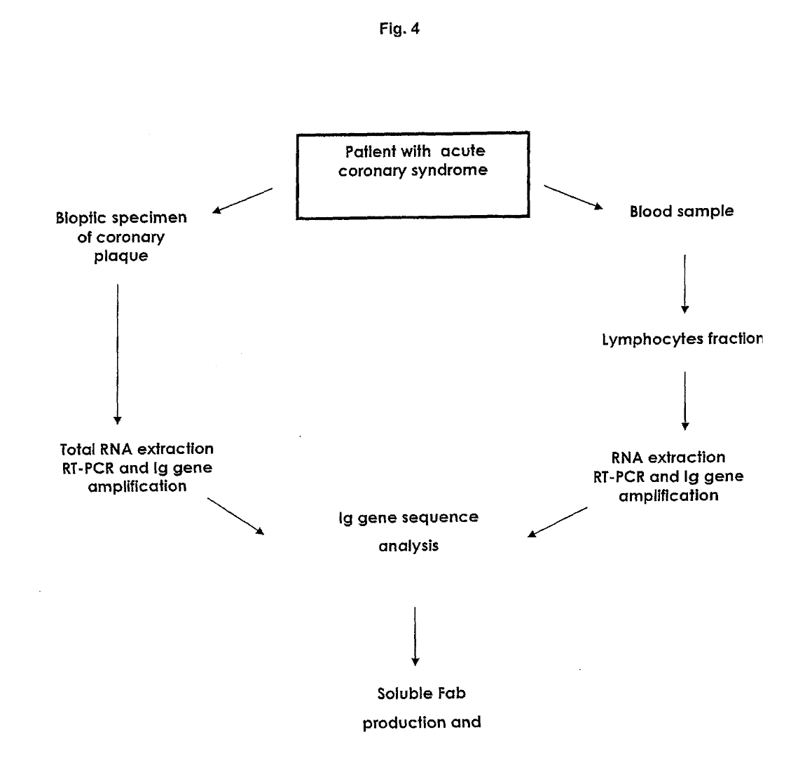

### DESCRIPTION OF THE INVENTION

### Definitions

In the present invention, and unless otherwise provided, the term “isolated polynucleotide” or “isolated nucleotide” refers to a polynucleotide molecule, wherein polynucleotidic and nucleotidic, respectively, and polynucleotide and nucleotide are used alternatively with the same meaning, which is substantially free of any other cellular material or component that normally is present or interact with it in its naturally occurring environment, such as fragments of other nucleotidic or polynucleotidic sequences, proteins or other cellular component. Unless otherwise provided, “complementary sequence” refers to the sequence which hybridizes to the sequence of interest under stringent conditions, resulting in two hydrogen bonds formed between adenine and tymine residues or three hydrogen bonds formed between cytosine and guanidine residues, respectively, and conservative analogs thereof having degenerative codon substitution or silent substitution, i.e. substitution of one or two or three consecutive nucleotides resulting in the same amino acid being coded due to the degeneracy of the genetic code.

The isolated polynucleotides within the meaning of the present invention, comprise, for instance, gene or gene fragments, exons, introns, mRNA, tRNA, rRNA, rybozyme, cDNA, plasmids, vectors, isolated DNA, probes and primers.

Unless otherwise indicated, the isolated polynucleotides of the invention, in addition to the specific ones described above, also comprise the complementary sequences thereto.

“cDNA” refers to the complementary DNA sequence, both single and double stranded and to any homologous sequence thereto and any fragment thereof, which codes continuously for an amino acidic sequence, i.e. its sequence is deprived of introns, and may be synthesized from isolated mRNA by retro-transcription techniques.

“Homologous sequence” within the meaning of the present invention refers to any sequence which is partially or almost identical to the sequence of interest; therefore, “homology” or “identity” of two or more sequences, comes from the factual measurement of the number of the same units, being those units nucleotides or amino acids, out of the total units componing said nucleotidic/amino acidic sequence, which occupy the same position. For example, 90% homology means that 90 of every 100 units making up a sequence are identical when the two sequences are aligned for maximum matching. Within the present invention, homologous sequences have an identity of at least 85%, preferably of 90%, more preferably of 95% and even more preferably of at least 97% or 99.5%.

“Conservative substitutions” of an amino acid is intended to be a substitution of an amino acid with another amino acid having the same properties, so that the substitution has no impact on the overall characterizing properties or functions of the peptide.

Examples of such conservative substitutions include the substitution of an amino acid with another amino acid belonging to the same group as follows:
(i) amino acids bearing a charged group, comprising Glutamic and Aspartic acid, Lysine, Arginine and Histidine;
(ii) amino acids bearing a positively-charged group, comprising Lysine, Arginine and Histidine;
(iii) amino acids bearing negatively-charged group, comprising Glutamic and Aspartic acid;
(iv) amino acids bearing an aromatic group, comprising Phenylalanine, Tyrosine and Tryptophan;
(v) amino acids bearing a nitrogen ring group, comprising Histidine and Tryptophan;
(vi) amino acids bearing a large aliphatic nonpolar group, comprising Valine, Leucine and Isoleucine;
(vii) amino acids bearing a slightly-polar group, comprising Metionine and Cysteine;
(viii) amino acids bearing a small-residue group, comprising Serine, Threonine, Aspartic acid, Asparagine, Glycine, Alanine, Glutamic acid, Glutamine and Proline;
(ix) amino acids bearing an aliphatic group comprising Valine, Leucine, Isoleucine, Metionine and Cysteine;
(x) amino acids bearing a small hydroxyl group comprising Serine and Threonine.

In the following disclosure, “CDR3” is a short sequence comprised within any of the heavy and the light region and refers to the complementary-determining region, which is formed by the junctions between the V-D-J gene (in the heavy chain) or V-J gene (in the light chain) segments coding for an antibody. CDR3 is found in the variable domains that complements an antigen.

Within SEQ ID NO:2 and 4, CDR3 regions are represented respectively by the following fragments: CVTLGRWSSWQGGALSW and CQQYHNWPPLTF (SEQ ID NO:9 and SEQ ID NO:10).

“Single clone” refers to a sequence coding for the CDR3 region of an antibody, which is able to specifically bind an antigen/epitope.

Sequences showing the same CDR3 are deemed to be produced by the same clone.

“Clonal variant” is intended to be any sequence, which differs by one or more nucleotide or amino acid, in presence of V region with identical mutations compared to the germline, identical VDJ or VJ gene usage, and identical D and J length.

“Replacement mutation” is intended to be a nucleotidic mutation which causes another amino acidic to be coded.

“Silent mutation” is intended to be a nucleotidic mutation which does not cause a change in the coded amino acid due to the degeneracy of the DNA.

An “expression vector” is intended to be any nucleotidic molecule used to transport genetic information.

An “isolated expression system” is intended to be a system for the expression of amino acidic molecules, and shall include one or more expression vectors comprising the nucleotidic sequences coding for one or more of the amino acidic molecules of the invention and a suitable host cell in which the one or more vectors are transfected.

“Host cell” as for the present invention is intended to be a cell comprising one or more expression vectors of the invention and which is capable of producing the corresponding coded amino acidic sequence or sequences or any fragments thereof, for example by expressing it on its surface.

“Antibodies” and “antibodies fragments” according to the present invention is intended to include whole antibodies, also referred to as immunoglobulin, of either type IgG, IgA, IgD, IgM or IgE, as well as any fragments thereof, such as proteolytic and/or recombinant fragments, like Fab fragments (produced upon digestion of Ig with papain), F(ab′)₂ (produced upon digestion of immunoglobulin with pepsin), Fab′, Fv, single chain antibodies (scFv) and single chain of antibodies, such as, for instance, heavy or light single chains.

“Ligand” within the present invention, is intended to be any agent that binds a recognized functional region of the antibody of the present invention or to any fragment thereof.

“Oligopeptide” according to the present invention is an amino acidic sequence comprising less than 50 amino acidic residues.

In the following description and unless otherwise provided, the “germline” sequence is intended to be the sequence coding for the antibody/immunoglobulin or of any fragment thereof deprived of mutations, therefore, the percentage of homology represents an indication of the mutational events which any type of heavy chain portion undergo after contact with an antigen; more in particular, said mutations involve the CDR3 portion of the antibody/immunoglobulin or of any fragment thereof.

The “R:S mutation” ratio refers to the ratio between replacing (R) and silent (S) mutations occurred in the FR or CDR3 portion of the antibody/immunoglobulin coding sequence.

Said ratio is higher for CDR3 than that of the FR sequence, as CDR3 undergoes an higher number of mutational event in order to adapt to the structure of the antigen. FR, in contrast, is a more conservative sequence, generally.

### P-Value

“P-value” represents the significance of a mutational event.

In particular, the process of somatic hypermutation of rearranged V segments and the antigen selection of mutants with a higher affinity, allow the affinity maturation, in order to generate antibodies with improved binding properties to the antigen. This process leads to an accumulation of replacement mutations (R) in CDR regions, which are directly involved in the binding of antigen. On the contrary the silent mutations (S) accumulate in the FR regions, which are usually more conservative sequences in order to maintain the conformation of the antibody. In absence of the antigen selection, a random mutational process results in random distribution of R and S mutations in the sequence of both heavy and light chains of an antibody. However during the selection process, the R:S mutation ratio for CDR3 is usually higher than that of the FR sequence. Therefore, the p-value, which is calculated by multinomial distribution model that the excess (as for CDR) or the scarcity (as for FR) of mutations occurred by chance, relates to the probability of an antigen selection process. A low p-value indicates that there is a high probability that the variability of the heavy and light chains compared to the corresponding germline sequence, is due to the antigen-driven affinity maturation of the antibody. A significant p-value is intended to be below 5%.

According to a first aspect the present invention provides a method for preparing antibodies comprising amino acidic sequences corresponding to SEQ ID NO:2 in the heavy chain variable region and SEQ ID NO: 4 in the light chain variable region, as well as the homologous sequences thereof, and any sequences bearing conservative substitutions and fragments thereof obtained by expression of polynucleotidic molecules. Preferably, said polynucleotidic sequences comprise SEQ ID NO: 1 and 3 and the complementary and homologous sequences thereto.

The polynucleotidic sequences of the present invention codes for the amino acidic sequences of antibodies or any fragments thereof which binds to an antigen or any fragment thereof possibly found in the coronary plaque.

Preferably, within the present invention, the isolated polynucleotides of the above embodiment are cDNA molecules, obtained by retro-transcription from mRNA molecules according to the well-known procedures in the art.

As indicated, these definitions are intended to encompass analogous sequences, so as to include those sequences wherein, in the case of amino acid sequences, at least one or more amino acids are substituted by a derivative, such as the corresponding D-isomer or, for example, the corresponding sulphated, glycosylated or methylated amino acid; or one or more and up to 10% of the total amino acids making up a sequence may be substituted by a derivative thereof, such as, for example, cysteine may be substituted by homocysteine. There are also included sequences bearing conservative substitutions.

According to the present invention, there are also included the polynucleotidic sequences coding for antibodies or for any fragments thereof according to the first embodiment of the invention and having homology of at least 80%, preferably of at least 90%, more preferably of at least 95% and even more preferably of at least of 97% compared to the germline, when using a database available in ImMunoGeneTics (available through the web site http://imgt.cines.fr).

In addition, as for the first object of the present invention, hypermutated amino acidic sequences are also encompassed.

Accordingly, there are also included the polynucleotidic sequences coding for the amino acidic sequences having a p-value of the CDR3 portion above mentioned less than 5%, preferably less than 2%, more preferably less than 1% and even more preferably less than 1‰ and the coded amino acidic sequences thereof.

As set hereinbefore, according to the present invention, there is included the synthesis of cDNA molecules, which is performed from mRNA isolated from a suitable sample of the active coronary plaque of a patient.

For the purpose of the present invention, said suitable samples of the active coronary plaque includes a sample of the coronary plaque taken immediately after an infarction event, i.e. so-called “fresh-sample” or, alternatively, a sample may be taken and conserved under liquid nitrogen for a suitable period of time so as not to impair nor alter its histological properties and be further analysed.

For the purpose of the present invention, patients with acute coronary syndrome (ACS) have been selected, which have experienced a typical chest pain occurring less than 48 hours from hospital admission or ECG changes suggesting myocardial damage. In order to exclude possible confusing factors, patients with recent infectious diseases, immunologic disorders, immunosuppressive therapy or neoplastic diseases have been excluded.

Isolation of mRNA molecules from the above suitable samples, i.e. both from coronary plaque and peripheral blood, is carried out according to well-known methods. For a reference, see, for instance Molecular cloning. Sambrook and Russell. Cold Spring Harbor Laboratory Press Cold Spring Harbor, N.Y. Third Edition 2001.

According to the second embodiment, the expression vector of the invention is selected from the group comprising for example, plasmid, cosmid, YAC, viral particle, or phage and comprises one or more of the polynucleotidic sequences according to the first embodiment of the invention; in a preferred aspect, the expression vector is a plasmid, comprising one or more of the polynucleotidic sequences according to the first embodiment of the invention.

In a most preferred embodiment of the invention, the expression vector, i.e. a plasmid, comprises the polynucleotidic SEQ ID NO:1 and 3. However, according to the degeneracy of the genetic code and to the use of preferential codons in each organism where recombinant expression is achieved, any polynucleotidic sequence encoding for SEQ ID NO 2 and 4 is deemed to be comprised within the scope of the present invention.

Expression vectors ordinarily also include an origin of replication, an operably linked, i.e. connected thereto in such a way as to permit the expression of the nucleic acid sequence when introduced into a cell, promoter located upstream the coding sequences, together with a ribosome binding side, an RNA splice site, a polyadenylation site and a transcriptional sequence. The skilled artisan will be able to construct a proper expression vector and, therefore, any proper expression vector according to the selected host cell; for example, by selecting a promoter which is recognized by the host organism.

In an even more preferred embodiment, the expression vector of the present invention is represented by the vector described by Burioni et al. (Human Antibodies 2001; 10 (3-4): 149-54).

The isolated expression system according to the invention may comprise a single expression vector comprising one or both the polynucleotidic sequences of the invention.

Alternatively, the above expression system may comprise two or more expression vectors, each of them comprising any one of the polynucleotidic molecules of the invention.

For example, an expression vector may comprise a polynucleotidic molecule of the invention coding for the light chain of an antibody or fragment thereof and a second expression vector may include a polynucleotidic molecule of the invention coding for the heavy chain of an antibody or fragment thereof.

In an embodiment of the invention, the expression system comprises a single expression vector including one or more of the polynucleotidic molecules comprising SEQ ID NO:1 to 3 and coding for the amino acidic sequences SEQ ID NO:2 to 4 or any fragment thereof with the same antigen specificity and any homologous sequence thereto.

In a preferred embodiment of the invention, the expression system comprises one expression vector comprising the polynucleotidic sequence coding for a light chain, i.e. SEQ ID NO: 3 and a second polynucleotidic sequence coding for a heavy chain, i.e. SEQ ID NO:1. Alternatively the expression system may comprise a suitable framework for the expression of CDR3 amino acid sequence SEQ ID NO: 9 or SEQ ID NO: 10, with the same specificity as the whole antibody. According to this embodiment, any nucleic acid sequence encoding SEQ ID NO:9 and 10 is comprised within the present invention, even though preferred sequences are those found within SEQ ID NO:1 and 3.

Preparation of the Expression Vector within the Expression System of the invention, includes the insertion of the appropriate nucleic acid molecule o molecules into one or more vector or vectors, which generally comprises one or more signal sequences, origins of replication, one or more marker genes o sequence, enhancer elements, promoters, and transcription termination sequences according to methods well-known in the art.

For a general reference to said procedure, see, for instance Phage display, Cold Spring Harbor Laboratory Press. Cold Spring Harbor, N.Y.

For instance, the sequences coding for the heavy chain of the present invention are inserted into the expression vector with a Flag o a six-Histidine tail, for easier detection or purification.

The host cell according to a fourth embodiment of the present may be, for instance, a prokaryotic cell or a eukaryotic cells.

Suitable prokaryotic cells include gram negative and gram positive and may include, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces. For example, publicly available strains which may be used are, for instance, E. coli K12 strain MM294 (ATCC 31,446); E. coli X1776 (ATCC 31,537); E. coli strain W3110 (ATCC 27,325) and K5 772 (ATCC 53,635) or E. coli XL1-Blue, which represents the preferred E. coli strain.

In addition to prokaryotes, eukaryotic microbes such as filamentous fungi or yeast are suitable host cells. Saccharomyces cerevisiae, also known as common baker's yeast, is commonly used; other yeast are, for instance, Saccharomyces, Pichia pastoris, or Kluyveromyces such as, for example, K. lactis, K. fragilis, K. bulgaricus, K. wickeramii, K. waltii, K. drosophilarum, K. thermotolerans, and K. marxianus, Schizosaccharomyces, such as Schizosaccharomyces pombe, yarrowia, Hansenula, Trichoderma reesia, Neurospora crassa, Schwanniomyces such as Schwanniomyces occidentalis, Neurospora, Penicillium, Tolypociadium, Aspergillus such as A. nidulans, Candida, Torulopsis and Rhodotorula.

In addition, suitable eukaryotic cells used for the preparation of the expression system may be derived from multicellular organisms as well, such as from invertebrate cells or plant cells. Plant cells include, for instance, Agrobacterium tumefaciens and Nicotiana tabacum. In addition, insect cells may be used, which include, for instance, Drosophila S2 and Spodoptera Sf9.

Conversely, mammalian host cell include Chinese hamster ovary (CHO) and COS cells. More specific examples further include monkey kidney CVI line transformed by SV40 (COS-7, ATCC CRL 1651); human embryonic kidney line, Chinese hamster ovary cells/−DHFR, mouse sertoli cells, human lung cells (W138, ATCC CCL 75); human liver cells (Hep G2, HB 8065); and mouse mammary tumor (MMT 060562, ATCC CCL51).

The selection of the appropriate host cell is deemed to be within the knowledge of the skilled person in the art, i.e. prokaryotic cells may be used for the preparation of antibodies fragments such as Fabs, while for the preparation of whole antibodies such as IgG, eukaryotic cells like yeasts may be employed. In the definition of antibody according to the present invention is comprised any scaffold suitable to express the light and/or the heavy variable chain or their specific CDRs, for example Fab fragments, F(ab′)2, Fab′, Fv and single chain antibodies (scFv).

Methods for cell transfection and transformation in order to prepare the above disclosed host cell comprising the above expression system depends upon the host cell used and are within the ordinarily knowledge of the skilled artisan. For example, treatments with calcium or electroporation are generally used for prokaryotes, while infection with Agrobacterium tumefaciens is used for transformation of certain plant cells. For mammalian cells, calcium phosphate precipitation may be used as disclosed by Graham and van der Eb, Virology, 52:456-457 (1978).

However, other methods for introducing polynucleotidic sequences into cells, such as, for example, nuclear microinjection, electroporation, bacterial fusion with intact cells, or polycations, may also be used.

Host cells, in addition, may also be transplanted into an animal so as to produce transgenic non-human animal useful for the preparation of humanized antibodies or fragments thereof. A preferred non-human animal includes, for instance, mouse, rat, rabbit, hamster.

The production of recombinant antibodies and fragments thereof according to an embodiment of the invention, is performed according to methods known in the art and includes the use of the isolated polynucleotidic sequences of the invention.

In particular, said method comprises the steps of:
a) preparing an expression system comprising one or more cDNA molecule or molecules encoding amino acid SEQ ID NO: 2 and 4 and preferably having SEQ ID NO:1 and 3, in any one of the above disclosed suitable host cells;
b) culturing the host cell under suitable growth conditions;
c) recovering and/or purifying the produced antibodies or any fragments thereof having amino acid SEQ ID NO: 2 and 4 or any fragments thereof.

In the method described above, fragments are preferably CDR3 fragments with SEQ ID NO: 9 CVTLGRWSSWQGGALSW and SEQ ID NO: 10 CQQYHNWPPLTF which may be expressed in suitable scaffold in any one of the above disclosed host cells. Suitable scaffold are usually derived from human antibodies where, in the variable region the pre-existing CDR3 is replaced by the CDR3 region of the invention. Minibodies or ScFv frameworks are also used for CDR3 expression.

The host cell comprising the expression system is grown under suitable growth conditions and the antibodies or any fragments thereof so produced, are then recovered and/or purified for further use.

In order to assess the influence on the results obtained by statistically occurring mutations or other mechanism different from those involved in the maturation of B-cells of the coronary plaque, cloning and sequencing is also performed on a small portion of a gene having a conserved region. Accordingly, as internal reference, β-globin gene is chosen; in particular, standard β-globin L48931 is used.

Therefore, it is a further object of the present invention, the isolated recombinant antibodies and fragments thereof produced by the host cell of the present invention and according to the method disclosed above, include immunoglobulin (shortly referred to as Ig) of the IgG type, while “fragments thereof” preferably include Fab fragments of IgG.

According to the present invention, there are also included the amino acidic sequences coding for antibodies or for any fragments thereof which may be produced according to the process above disclosed and having homology of at least 80%, preferably of at least 90%, more preferably of at least 95% and even more preferably of at least of 97% compared to the germline, when using a database available in ImMunoGeneTics (available through the web site http://imgt.cines.fr).

In addition, there are also included the amino acidic sequences having a p-value of the CDR3 portion less than 5%, preferably less than 2%, more preferably less than 1% and even more preferably less than 1‰.

A further embodiment of the invention is represented by an assay, preferably an immunoassay, comprising the use of antibodies or of any fragments thereof, according to the present invention.

Immunoassays are test based on the formation of an antigen/antibody complex and can be either competitive or non-competitive.

Competitive immunoassays include the testing of unknown samples containing a particular antigen which competes for the binding to the antibodies with another, preferably labelled antibody; therefore, the response is inversely proportional to the concentration of the antigen in the unknown sample.

Conversely, non-competitive immunoassays, also called “sandwich assays”, include the use of an immobilized antibody as disclosed in the present invention, bound by an antigen, thus forming a complex which is targeted by a labelled antibody; the result of said methods is, therefore, directly proportional to the concentration of the antigen.

Widespread used immunoassays include, for example, RIA (Radio Immuno Assay), Western Blot, ELISA (Enzyme-linked Immunosorbent Assay), immunostaining, immunoprecipitation, immunoelectrophoresis, immunofluorescence, luminescent immunoassay (LIA), immunohystochemistry, which are routinely used in lab practise.

A preferred immunoassay according to the present invention is an ELISA test. ELISA is a well-established biochemical technique, which allows the detection and further quantification of biomolecules, such as antibodies or fragments thereof, antigens, proteins, hormones and other organic molecules, in a given sample; preferably, according to the present invention, the above mentioned ELISA test is used for the detection of a specific antigen, using antibodies or fragments thereof comprising SEQ ID NO: 2 and 4 or alternatively SEQ ID NO: 9 and 10.

ELISA test, in particular, may include the use of two antibodies, one of which, is selective for the molecule of interest, the antigen, immobilized onto an ELISA plate. This is preferably one of the antibodies according to the present invention. A mixture possibly containing a molecule of interest, even comprised within a complex matrix such as a cell or tissue lysate, is added, incubation for a suitable and sufficient time is allowed, then a first washing is performed in order to remove unbound material. The secondary antibody coupled to an enzyme and specific for the complex formed between the molecule of interest and the first antibody is further added. There follows a second step of washing of the ELISA plate and the addition of a chromogenic substrate. The resulting variation in colour may be assessed by spectrophotometric techniques and is directly related through a colorimetric standard curve to the quantity of the complex formed and thus to the concentration of the molecule of interest present in the sample.

Samples to be tested by the above immunoassay of the invention are, for example, samples of unstable coronary plaque isolated from patient immediately after an infarction event, i.e. a so-called “fresh” sample as said before, or a sample which has been conserved under liquid nitrogen after being taken; alternatively, the sample may consist of whole blood, serum, cultured cell i.e. cell lines or their lysates. Useful cell lines are for example, Hep2 (ATCC number CCL-23), U87 (ATCC number HTB14) MRC5(ATCC number CCL-171) either infected by pathogens, i.e. viruses such as CMV or not infected, or molecular libraries exposing synthetic peptides or libraries exposing pathogenic agent antigens. The use of cell lines for antibody screening has been described for example in Sack U. et al. Ann N Y Acad. Sci. 2009 September; 1173:166-73.

Immunohystologic assay can be also performed in order to investigate the presence inside the plaque of the ligands identified and disclosed in the present invention according to the above embodiments.

As said above, the immunoassays of the present invention may be designed to competitively identify antigens involved in the development of the atherosclerotic plaque linked to a coronary disease.

The immunoassay according to the present invention represents an extremely important tool for the identification of antigens in a atherosclerotic plaque and it may represent a valuable diagnostic or prognostic tool in the screening of the population at risk of developing acute coronary syndrome (ACS) or other coronary diseases.

According to a further embodiment of the invention, there is disclosed a therapeutic composition comprising the antibodies or any fragments thereof of the present invention and a therapeutic moiety covalently linked thereto. Said therapeutic composition selectively target a therapeutic agent to a coronary atherosclerotic plaque site.

Well-known advantages of said targeted composition include, among others, the possibility of reducing the quantity of active principle to be administered, thus reducing the potentially side effects thereof.

For said purpose, therapeutic moieties may include, as non limiting examples, radionuclides, drugs, prodrugs, hormones, hormone antagonists, receptor soluble receptor, receptor antagonists, enzymes or proenzymes activated by another agent, cytokines, antimicrobial agents or toxins

A further embodiment of the invention relates to a diagnostic composition comprising the antibodies of the invention or any fragment thereof linked to a diagnostic moiety for the visualisation of the coronary plaque site.

The diagnostic composition according to the present invention comprises the antibody or any fragments thereof according to the present invention, covalently linked to at least one diagnostic moiety able to selectively target the coronary plaque site allowing its localization.

Therefore, it will be possible to precisely localise the site where the coronary plaque developed and to even better understand the extent of the occurred lesion to the vase. In addition, it may represents a very useful tool before removal of the plaque by surgery.

Diagnostic moieties allow the detection by the visualising techniques used in the field of medicine, such as, for example, MRI (magnetic resonance imaging), CT (computer tomography), ultrasound, ecography, x-rays, and other diagnostic techniques within the knowledge of the skilled person in the art. The diagnostic moiety will be selected according to the diagnostic technique of choice.

According to one of the embodiment of the immunoassay of the present invention has lead to the identification of ligands, defined as agents that bind any surface or internal sequences or conformational domains or any other part of the target antibody or fragments thereof and may encompass agents that have no apparent biological function, beyond their ability to bind the target of interest.

In a preferred embodiment, the ligand of the present invention is an oligopeptide, preferably comprising 4 to 12 amino acids, more preferably is a peptide comprising 4 to 10 and even more preferably is a peptide comprising 6 to 8 amino acids, within amino acid SEQ ID NO: 5-8 (artificial antigens) or corresponds to peptides with said sequence.

Alternatively said peptides may be used in immunoassays to competitively identify antigens involved in the development of the atherosclerotic plaque linked to a coronary disease.

With the aim of better understanding of the present invention, and without representing any limitation to it, the following Examples are given.

### Experimental Part

### Example 1

### Sample Collection

A sufficient amount of tissue (usually about 1-2 milligrams) is obtained from an atherosclerotic plaque of a patient with acute coronary syndrome undergoing coronary atherectomy and stored in liquid nitrogen.

### Example 2

### mRNA Extraction

The plaque taken according to Example 1 is homogenized and the total mRNA is extracted according to conventional methodologies using a commercial kit for the extraction of mRNA (Rneasy kit, Qiagen, Germany) and according to the instructions provided by the manufacturer.

### Example 3

### mRNA Retrotranscription

Reverse transcription of mRNA from the coronary plaque sample obtained as from Example 2, with the patient's informed consent, is performed using a commercial kit for the retrotranscription of mRNA, Superscript III RT (Sigma-Aldrich, St Louis, Mo.) according to the manufacturer's instruction. The cDNA synthesis is performed according to standard procedures from the total mRNA primed with oligo(dT).

### Example 4

### Amplification of cDNA Sequences

1 μl of cDNA obtained from the Example 3 is used for polymerase chain reaction. The reverse primers are designed in order to anneal to the segments of sequences coding for the constant region of heavy and light chains respectively. The forward primers are “family specific” and are designed to correspond to the 5′ end of the heavy and light chain genes in the first framework region; see, as a reference, Phage display, Cold Spring Harbor Laboratory Press. Cold Spring Harbor, N.Y. Third Edition 2001. For the heavy chains, primers specific for IgG1 and IgG2 isotypes are used, whereas for the light chains primers specific for K isotype are used. Amplification round is conducted with the following thermal profile: 94° C. for 15 seconds, 52° C. for 1 minute and 72° C. for 90 seconds. The reaction is conducted for 35 cycles. A negative control (the same mixture without DNA) and a positive control (a known sequence is inserted) are included in each reaction. The PCR product is analyzed by electrophoresis in a 2% agarose gel containing ethidium bromide. The reaction yields a ≅650 bp band corresponding to the light chains, and a 700 bp corresponding to the heavy chains. The amplicon, i.e. the product of the PRC process) is extracted from the gel with the use of a commercial kit for the extraction of DNA (QIAquick gel extraction kit; Qiagen, Germany) according to the manufacturer's instructions. Finally, the PCR products are recovered as per standard methods.

### Example 5

### Molecular Cloning and Combinatorial Antibody Fab Fragment Phage-Display Minilibrary

The PCR products of heavy and light chains that make the Fab (variable region and the CH1 domain) amplified from a human coronary plaque according to the previous Examples are cloned into a phagemidic vector (pRB32) that allows the combinatorial generation o heavy and light chain pairs and exposes (phage display) onto the external phage surface the Fab fragment codified by the DNA contained into the viral particle. This is obtained by fusing the heavy chain fragment with a phage M13 membrane protein.

This allowed the generation of a combinatorial antibody Fab fragment phage-display minilibrary.

### Example 6

### Cell Lysate Preparation

Infected and unifected MRC5 and Hep-2 cell lysates are obtained after 5 days after infection with HCMV (MOI 0,1). Hep-2 (ATCC CCL-23) cells are grown in E-Mem (Invitrogen 0820234DJ) supplemented with Antibiotic/Antimycotic Solution (Invitrogen, Antibiotic/Antimycotic Solution, liquid 15240-062) and 10% FBS. Cells are regularly split 1:10 every 5 days. Five million cells are washed in PBS and lysed by using RIPA buffer (50 mM Tris HCL pH 8+150 mM NaCl+1% NP-40+0.5% NA deossicolate+0.1% SDS).

### Example 7

### Immunoaffinity Selection by Biopanning on Cell Lysate (Hep-2), Carotid Lysate, Artificial Antigens

The night before biopanning, coat an ELISA plate O/N at 4° C. with 50 μL/well of antigen(s) (100 ng/well) solution in coating buffer (0.1 M Bicarbonate pH=8.6). After washing with deionised H₂O wells are blocked completely with PBS/BSA 3%, sealed and incubated in a humidified container for 1 hour at 37° C. Fifty μL phage suspension are added to each well (total of about 10¹¹ PFU) and incubated for at least 2 hours at 37° C. after sealing the plate. Phage is removed from every well (and kept for titration), washed 10 times with PBS/Tween 0.05%. After intensive washing, phages bound to the antigen are eluted by washing each well with 50 μl of Elution Buffer (0.1M HCl, adjusted with glycine to pH=2.2, BSA 1 mg/ml) and adding to the eluent 3 μl of 2M Tris base. After the elution, 2 ml of fresh XL-1 blue are infected with the eluted phage. After an incubation at 37° C. for 15 min, 10 ml of 37° C.-prewarmed Superbroth (SB) (20 μg/ml ampicillin and 10 μg/ml tetracycline) are added. After incubation for 1 hour at 37° C. on a shaker, 100 ml of SB containing 100 μg/ml Amp and 10 μg/ml Tet are added to each 10 ml-culture and incubated for 1 hour at 37° C. on a shaker. An helper phage VCS M13 (total of 1012 PFU) is used to infect the culture and incubated on the shaker for 2 hours at 37° C. After addition of kanamycin (μg/ml) culture are incubated on a shaker O/N at 30° C.

The day after cells are spun down at 6000 RPM (Sorvall SS34), 4° C. for 15 minutes and PEG8000 (Sigma-Aldrich) is added to the supernatant to a final volume of 20 ml. Phages are let precipitate on ice for 30 minutes and then centrifuged at 11,000 RPM (Sorvall SS34) for 20 min. at 4° C.). Phages are then resuspended in 2 ml PBS/BSA 1%, and stored at 4° C. for subsequent biopanning rounds.

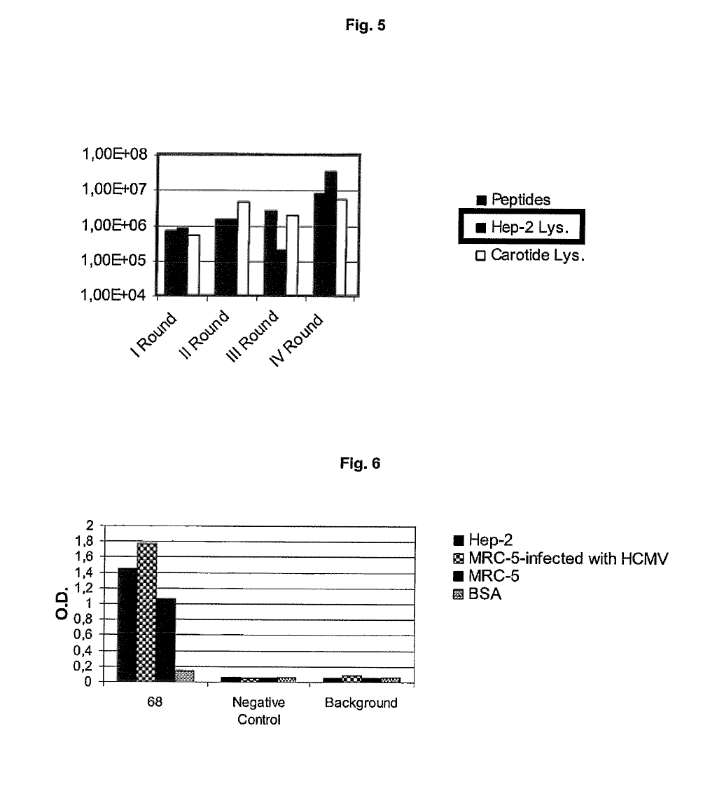

After each elution and at every round of selection, eluted phages are titered by plating 1 μl and 0.1 μl on LB plates (10-4 and 10-5 dilution of total) of the phage infected bacterial population. By titrating eluted phages after each round of selection, one can evaluate the efficacy of the enrichment procedure. The procedure is repeated four times allowing enrichment of selected population. Results are shown in FIG. 5.

### Example 8

### Analysis of Selected Clones after Biopanning Procedure on Hep-2 and Artificial Antigens

In all bio-panning procedures, the Fab of the invention is selected and it is predominant in the recovered phage population after the fourth round.

### Example 9

### Sequencing

The clones obtained according to the previous Examples are sequenced for quantitative and qualitative analysis.

### 9.1) Heavy and Light Chain Sample Processing

A sample of clones of heavy and light chains obtained from coronary plaque sample obtained according to Example 4, is picked up in order to be sequenced by Big Dye chemistry and analyzed using ABI PRISM 3100 sequencer.

The obtained sequences are individually aligned to the germline segments using a database available in ImMunoGeneTics (available through the web site http://imgt.cines.fr), in order to identify the V, D, J and V and J genes recurrence as for the heavy and light chains respectively, the homology level with the germline and the extent of somatic mutations. CDR3 sequence identity is used for identifying the clones; as mentioned above, sequences with identical CDR3 are deemed to derive from the same clone.

The polynucleotidic sequence from coronary plaque sample obtained according to the above Example 4 for the heavy chain correspond to SEQ ID NO: 1 and coding for the amino acidic sequence corresponding to SEQ ID NO: 2. The polynucleotidic sequence from coronary plaque sample obtained according to the above Example 4 for the light chain correspond to SEQ ID NO: 3 and coding for the amino acidic sequence corresponding to SEQ ID NO: 4.

### 9.2) β-Globin Sequence: Internal Reference

The analysis of five clones shows that the obtained sequence of β-globin is identical to the sequence present in database, thus demonstrating that no mutational event was introduced by the process.

9.3) Light Chain from Coronary Plaque Sample

The results of clone sequencing from the coronary plaque sample according to Example 4 are shown in the following Table I for each clone V, D and J gene column report the type of sequence found to code for the V, D and J variable portion of the heavy chain, respectively. Homology percentage refers to the percentage of homology between the sequence cloned from the coronary plaque sample and the sequence of the corresponding germline sequence as above disclosed.

TABLE I

| # | VK | JK | Homology | R:S mutations | | p-value | |
|---|---|---|---|---|---|---|---|
| Clone | gene | gene | (%) | FR | CDR | FR | CDR |
| 8 | V3-15*01 | J4*01 | 94.90 | 3/2 | 5/2 | 0.00197 | 0.00091 |

9.4) Heavy Chain from Coronary Plaque Sample

The same procedure adopted for the analysis of the sequences of the light chains is repeated for the sequence of the heavy chains obtained according to Example 4.

The results are shown in the following Table II.

TABLE II

| # | VK | DK | JK | Homology | R:S mutations | | p-value | |
|---|---|---|---|---|---|---|---|---|
| Clone | gene | gene | gene | (%) | FR | CDR | FR | CDR |
| 6 | V5-51*03 | D6-13*01 | J5*01 | 82.95% | 20/16 | 8/1 | 0.0047 | 0.1848 |

Therefore, as clone 6 of the sequence amplified from the plaque shows a higher divergence from the germline sequence, this has been selected for expression together with light chain 8.

### 9.5) Results

The above data show that both heavy and light chain from coronary plaque sample have an oligoclonal pattern and a characteristic VDJ and VJ gene pattern, respectively.

### Example 10

### Preparation of the Expression System with Sequences from Coronary Plaque Sample and Transformation of Host Cells

Clone 8 of the light chain (corresponding to SEQ ID NO: 3) and clone 6 of the heavy chain (corresponding to SEQ ID NO: 1) of the coronary plaque sample are selected to be transfected into the expression vector for the preparation of the soluble Fab fragments according to the following procedure.

Gene encoding for the light chain selected according to the above Example 6 and corresponding to SEQ ID NO: 3 is transferred into the expression vector pRB/expr. In the expression vector comprising the gene coding for the selected light chain is further introduced the gene coding for the heavy chain corresponding to the clone 6, SEQ ID NO: 1 with the method disclosed in Burioni et al. Hum Antibodies. 2001; 10(3-4):149-54.

The expression vector is introduced into the E. coli XL-1 Blue for the expression of soluble Fabs.

In particular, 10 ml of SB (Super Broth, Becton, Dickinson, N.J.) with ampicillin (100 ng/ml, Sigma-Aldrich, St Louis, Mo.) and tetrayicline (10 ng/ml, Sigma-Aldrich, St Louis, Mo.) is inoculated with a single bacterial colony from a fresh plate and incubated overnight at 37° C. in an orbital shaker After that, 2.5 ml of this colture is inoculated into 1 liter of SB/amp-tet (the above mixture of SB, ampicillin and tetracyclin) into a 5 liter flask and allowed to grow until an Optical Density (OD₆₀₀) of approximately 1.0. Then IPTG (isopropyl-beta-D-thiogalactopyranoside; Biorad, California) is added up to a final concentration of 1 mM and the bacterial culture are incubated overnight at 30° C. in the orbital shaker. Thus, bacteria are centrifuged at 3000 rpm for 20 minutes at 4° C. and the pellets are resuspended in 10 ml PBS. Subsequently, 50 μl PMSF (from a stock solution of 100 mM) is added in order to inhibit the proteases and bacteria are sonicated three times in ice, 3 minutes for each run. The bacterial culture is centrifuged at 18000 rpm for 45 minutes at 4° C. and the supernatant is filtered carefully with a 0.22 μm diameter membrane (Millipore®). Meanwhile, the column is washed with 10 volumes of PBS and subsequently the filtered supernatant is added slowly to the column. After washing with at least 30 volumes of PBS, Fabs are eluted with 100 mM glycine/HCl pH 2.5. 10 fractions are collected (each one of about 1 ml) and immediately neutralized with Tris 1M pH 9.

Purified Fabs are tested in SDS-PAGE gel in non-reducing conditions showing a single band of approximately 50 kDa.

Fabs are quantified comparing the relative band with at least two different standard concentrations of BSA.

### Example 11

### Immunohistology Assay

A fresh sample of plaque is frozen in liquid nitrogen and sectioned using a cryostat. Sections 5 μm thick are fixed with ice-cold acetone and blocked with a serum blocking solution (2% serum, 1% BSA, 0.1% Triton X-100, 0.05% Tween 20) for 1 hour at room temperature. The fixed sections are probed with the Fab produced and identified according to the present invention, at an appropriate dilution, and incubated for 2 hours at room temperature. Sections are washed five times with PBS and an appropriate dilution of a FITC (fluorescein isothiocyanate)-conjugated secondary anti-human Fab (Sigma-Aldrich, St Louis, Mo.) is added. After 30 minutes at room temperature, sections are washed again and the complex ligand/antibody thus formed is detected with a fluorescence microscope.

### Example 12

### Antibody Screening of Phage Library

Panning of the random p hag e-displayed peptide library expressing dodecapeptides at the N-terminus of cpIII coat protein of the filamentous phage M13 (Ph.D.-12™ Phage Display Peptide Library Kit, Catalog #E8110S, New England Biolabs, Beverly, Mass.) is performed according to the manufacturer's instructions using Fab-coated high-binding 96-well ELISA plates (Costar 96w polystyrene ½ area flat bottom HI-binding flat bottom, cat #3690). In order to remove phages binding to antibody conserved regions, a negative selection is performed from the second round of panning by combining the amplified phages with 25 μg of a pool of human standard IgG (Endobulin, A.T.C J06BA02, Baxter S.p.A.) for 1 hour at 37° C.

Four rounds of selection are performed as described above, panning the amplified phage on Fabs produced and identified according to the present invention and the same pool of standard IgG used for the negative selection.

### Example 13

### Peptide Screening and DNA Sequence Analysis

All the phages obtained as from Example 12 are used to infect E. coli strain ER2537 and randomly picked single plaques are screened in enzyme-linked immunoassay on Fabs produced, identified according to the present invention adsorbed on a solid phase and the pool of standard IgG.

Antigen-coated plates (Costar 96w polystyrene ½ area flat bottom HI-binding flat bottom, cat #3690) are washed and blocked with a solution of PBS/BSA 1% for 1 hour at 37° C.; 50 μl of 10⁸ phages per milliliter are added and incubated for 2 hours at 37° C.

Plates are washed 10 times with PBS (0.1% Tween-20; Sigma-Aldrich, St Louis, Mo.); afterward, 50 μl of a 1:3000 dilution in PBS of a HRP-conjugated anti-M13 antibody (GE Healthcare 27-9411-01) is added.

After 2 hours at 37° C. plates are washed P with PBS (0.5% Tween-20; Sigma-Aldrich, St Louis, Mo.), specific bound phages are detected by adding 100 μl of substrate (Sigma-Aldrich, St Louis, Mo.) and plates are read for an Optical Density of 450 nm after 30 minutes at room temperature.

Positive clones showing an OD₄₅₀ₙₘ value >1 on Fabs of the present invention and OD₄₅₀ₙₘ value <0.3 on pool of IgG are scored as positives and evaluated by sequence analysis using the software Pepitope http://pepitope.tau.ac.il/index.html. From peptide sequence analisys conserved aminoacidic positions are identified and four peptides are selected on the basis of the amount of consensus residues present in their sequences.

Four peptides have been identified, and the related sequences corresponding to SEQ ID NO: 5 to 8.

### Example 14

### Enzyme-Linked ImmunoSorbent Assay

Elisa plates (Costar 96 wells polystyrene ½ area flat bottom HI-binding flat bottom, cat #3690) are coated with serial dilution of Hep-2 Lysate, MRC5 and MRC5 infected with HCMV (1000 ng, 200 ng, 40 ng and 8 ng in PBS, quantified by a standard BCA assay, Pierce) overnight at 4° C. After blocking with PBS+BSA3% for 2 hours at 37° C., serial dilutions of Fab 68 (20 μg/ml, 10 μg/ml, 5 μg/ml, 2.5 μg/ml, are incubated with the coated antigens for 1 hour at 37° C. After washing with PBS +Tween20 0.1% (SIGMA cod. PL379), plates are incubated with anti human IgG peroxidase (SIGMA cod. A2290) for 30 minutes at 37° C. After washing with PBS+Tween 0.1%, TMB substrate was added to the wells (PIERCE TMB substrate kit for peroxidase cod. SK 4400). ELISA plates are analysed with a spectrophotometer at 450 nm. Results are shown in FIG. 6.

### Example 15

### Immunofluorescence with Fab 68 on Human Fibroblasts

Human Cytomegalovirus (HCMV) reference strain AD169 is propagated on MRC-5 fibroblasts (BioMerieux, Lyon, France). Confluent MRC-5 cells are conserved on Eagle's minimum essential medium (Invitrogen) containing 2% fetal calf serum (Sigma). For immunofluorescence staining human fibroblasts are seeded onto 24-well plates containing sterile glass coverslips. When cell cultures are confluent as monolayers, cells are infected as follows: medium is eliminated and cell monolayers are infected at high multiplicity of infection (about MOI 0,1) with HCMV. Plates are incubated at 37° C. in the presence of 5% CO₂ for 96 hours. After incubation, media are removed and cells are fixed by 4% para-formaldehyde solution for 8 minutes. After washing with PBS solution cells are permeabilized with TritonX100 (0.1% solution, SIGMA T-8787) and rinsed with PBS. After drying, cells are incubated with Fab68 and an unrelated negative control Fab for 1 hour at 37° C. in a humid atmosphere. After washing with PBS, fixed cells are incubated with anti-human IgG Fab specific FITC-Conjugate (Sigma) for 30 minutes at 37° C. in a humid atmosphere. The glass are mounted with glycerol buffer and observed by MRC 1024 Laser Scanning Confocal microscope (Biorad).

### Example 16

### Peptide Synthesis

### 12.1) General

Abbreviations for Chemical Reagents, Chemical Structure Moieties and Techniques: AA-amino acid, AcOH-Acetic acid, ACN-Acetonitrile, API-ES-Atmospheric pressure ionization electrospray, Btn-Biotin, Boc-tert-Butyloxycarbonyl, DCM-Dichloromethane, DIC-N,N-Diisopropylcarbodiimide, DIEA-N,N-Diisopropylethylamine, DMF-N,N-Dimethylformamide, Et₂O-Diethyl ether, Fmoc-9-Fluorenylmethoxycarbonyl, Adoa-8-Amino-3,6-dioxaoctanoic acid, HFIP-1,1,1,3,3,3-hexafluoro-2-propanol, HOBt-N-Hydroxybenzotriazole, MeOH-Methanol, Neg. ion-Negative ion, NHS-N-Hydroxysuccinimide, NMP-N-Methylpyrrolidone, Pip-Piperidine, Pos. ion-Positive ion, HBTU-O-(Benzotriazol-1-yl)-N,N,N′,N′-tetramethyluronium hexafluorophosphate, PyBOP-Benzotriazole-1-yl-oxy-tris-pyrrolidino-phosphonium

hexyluorophosphate, t_{R}-Retention time (minutes), Reagent B (88:5:5:2—TFA:H₂O:phenol:TIPS—v/v/wt/v), Su-Succinimidyl, TFA-Trifluoroacetic Acid, TIPS-Triisopropylsilane, H₂O-Water.

Peptide amino acid sequences are disclosed in Table V.

Solvents for reactions, chromatographic purification and HPLC analyses are E. Merck Omni grade solvents from VWR Corporation (West Chester, Pa.). NMP and DMF are purchased from Pharmco Products Inc. (Brookfield, Conn.), and are peptide synthesis grade or low water/amine-free Biotech grade quality. Piperidine (sequencing grade, redistilled 99+%) and TFA (spectrophotometric grade or sequencing grade) are purchased from Sigma-Aldrich Corporation (Milwaukee, Wis.) or from the Fluka Chemical Division of Sigma-Alrich Corporation. Phenol (99%), DIEA, DIC and TIPS are purchased from Sigma-Aldrich Corporation. Fmoc-protected amino acids, PyBop, and HOBt used are purchased from Nova-Biochem (San Diego, Calif., USA), Advanced ChemTech (Louisville, Ky., USA), Chem-Impex International (Wood Dale III, USA), and Multiple Peptide Systems (San Diego, Calif., USA). Fmoc-Adoa and Btn-Adoa-Adoa-OH are obtained from NeoMPS Corp (San Diego, Calif.).

Analytical HPLC data are generally obtained using a Shimadzu LC-10AT VP dual pump gradient system employing either Waters X-Terra® MS-C18 (5.0μ, 50×4.6 mm; 120 Å pore size) or Waters Sunfire™ OBD-C8 (4.6×50 mm 3.5μ, 120 Å pore size) columns and gradient or isocratic elution systems using H₂O (0.1% TFA) as eluent A and ACN (0.1% TFA) as eluent B. Detection of compounds is accomplished using UV at 220 and/or 230 nm. Preparative HPLC is conducted on a Shimadzu LC-8A dual pump gradient system equipped with a SPD-10AV UV detector fitted with a preparative flow cell. Generally the solution containing the crude peptide is loaded onto a reversed phase Waters Sunfire™ OBD C8 (50×250 mm; particle size: 10.0μ, 120 Å pore size) column, using a third pump attached to the preparative Shimadzu LC-8A dual pump gradient system. After the solution of the crude product mixture is applied to the preparative HPLC column the reaction solvents and solvents employed as diluents, such as DMF or DMSO, are eluted from the column at low organic phase composition. Then the desired product is eluted using a gradient elution of eluent B into eluent A. Product-containing fractions are combined based on their purity as determined by analytical HPLC and mass spectral analysis. The combined fractions are freeze-dried to provide the desired product.

Peptide characterization has been carried out by mass-spectrometry on an Agilent LC-MSD 1100 Mass Spectrometer, using API-ES in positive ion mode. Generally the molecular weight of the target peptides is ˜2000. Pure peptide preparations have been obtained by HPLC purification.

### 12.2) General Methods for Solid Phase Peptide Synthesis (SPPS)

The linear peptides are synthesized by an established automated protocol on a Rainin PTI Symphony® Peptide Synthesizer (twelve peptide sequences/synthesis) using Fmoc-Pal-Peg-PS resin (0.2 mmol/g) and/or suitably preloaded resins, Fmoc-protected amino acids and PyBop-mediated ester activation in DMF.

### 12.3) Purification of Peptides—General Procedure

The crude peptide (˜200-500 mg) is purified in one run by reversed phase HPLC. The crude peptide (˜200 mg) dissolved in ACN (10 mL) is diluted to a final volume of 50 mL with H₂O and the solution is filtered. The filtered solution is loaded onto a preparative HPLC column (Waters, Sunfire™ Prep C8, 50×250 mm 10μ, 120 Å) which had been pre-equilibrated with 10% ACN in H₂O (0.1% TFA). Product-containing fractions of >95% purity are combined and freeze-dried to afford the corresponding peptide. After isolation, the peptides are analyzed by HPLC and mass spectrometry to confirm identity and purity. Data for peptides are provided in Table V (Sequence and Yield).

TABLE V
Peptide Sequence, yield and identification
number in sequence listing

| | | Yield in | SEQ ID |
|---|---|---|---|
| CPD# | Sequence | mg (%) | NO |
| 1 | Ac-TMGFTAPRFPHY-NH₂ | 167.0 | 5 |
| | | (46%) | |
| 2 | Ac-MQSPFTPHFAER-NH₂ | 137.0 | 6 |
| | | (38%) | |
| 3 | Ac-MQSPIVLPLSLS-NH₂ | 131.0 | 7 |
| | | (41%) | |
| 4 | Ac-HHEPGAWLPLSP-NH₂ | 209.0 | 8 |
| | | (62%) | |

1. A recombinant antibody comprising amino acid SEQ ID NO:2 and 4.

2. A process for the preparation of the recombinant antibody according to claim 1 comprising the steps of:
a) preparing an expression system for a host cell comprising two polynucleotidic molecules encoding SEQ ID NO:2 and 4 or any fragment thereof;
b) culturing said host cell under suitable growth conditions;
c) recovering and purifying an antibody comprising SEQ ID NO:2 and 4, or any fragment thereof.

3) The process of claim 2 further comprising a step d) where the binding to a sample selected from the group consisting of: an isolated atherosclerotic plaque, a cell line, a cell line lysate and a biological sample, is determined.

4) The process of claim 3 wherein said cell line is selected in the group consisting of: Hep2 (ATCC number CCL-23), U87 (ATCC number HTB14) and MRC5 (ATCC number CCL-171), each either infected or not infected by pathogens.

5) The process of claim 2 where in step c) said fragments are respectively SEQ ID NO 9 or 10.

6) The process of claim 2 wherein the host cell is selected from the group consisting of prokaryotic, yeast and eukaryotic cells.

7) The process according to claim 6 wherein prokaryotic cells are selected from the group consisting of Enterobacter, Escherichia, Erwinia, Klebsiella, Proteus, Salmonella, Serratia, Shigella, Bacilli, Pseudomonas, Streptomyces, E. coli, Salmonella typhimurium, Serratia marcescens, Bacillus subtilis, Bacillus licheniformis, and Pseudomonas aeruginosa.

8) The process according to claim 6 wherein yeast cells are selected from the group consisting of Saccharomyces, Pichia pastoris, Kluyveromyces such as K. lactis, K. fragilis, K. bulgaricus, K. wickeramii, K. waltii, K. drosophilarum, K. thermotolerans, K. marxianus, Schizosaccharomyces, such as Schizosaccharomyces pombe, yarrowia, Hansenula, Trichoderma reesia, Neurospora crassa, Schwanniomyces such as Schwanniomyces occidentalis, Neurospora, Penicillium, Tolypociadium, Aspergillus such as A. nidulans, Candida, Torulopsis and Rhodotorula.

9) The process according to claim 6 wherein eukaryotic cells are selected from the group consisting of Chinese hamster ovary (CHO), monkey kidney CVI line transformed by SV40 (COS-7, ATCC CRL 1651), human embryonic kidney line, Chinese hamster ovary cells/-DHFR, mouse sertoli cells, human lung cells (W138, ATCC CCL 75); human liver cells (Hep G2, HB 8065); mouse mammary tumor (MMT 060562, ATCC CCL51), plant isolated recombinant host cells tabacum; and insect recombinant isolated cells.

10) A method for detecting antigens involved in the development of an atherosclerotic coronary plaque linked to a coronary disease comprising performing an immunoassay using the antibody of claim 1.

11) The method according to claim 10 wherein said immunoassay is selected in the group consisting of: RIA (Radio Immuno Assay), Western Blot, ELISA (Enzyme-linked Immunosorbent Assay), immunostaining, immunoprecipitation, immunoelectrophoresis, immunofluorescence, luminescent immunoassay (LIA), and immunohystochemistry.

12) The method according to any one of claims 10-11 wherein detection of an antigen in a sample isolated from a patient is indicative of a risk of developing an acute coronary syndrome (ACS).

13) A method for identifying a ligand which binds to the recombinant antibodies according to claim 1, comprising the steps of:
a) binding said antibodies or any fragment thereof onto a solid phase;
b) removing unbound material by one or more washing steps;
c) contacting a candidate ligand with the solid phase prepared in step a) and allowing incubation of said candidate ligand and the solid phase for a suitable period of time;
d) removing unbound material by one or more washing steps;
e) adding a secondary antibody specific for the complex of the antibody of step a) with the candidate ligand bound thereto; and
f) identifying the bound ligand to the antibodies of step a).

14) The method according to claim 13 wherein the candidate ligand is comprised within a biologic sample.

15) The method according to claim 14 carried out ex-vivo or in vitro wherein said sample is selected from whole blood, serum, coronary plaque biopsies whole cells and lysates thereof.

16) The method according to claim 13 wherein the candidate ligand is comprised within a molecular repertoire library.

17) The ex-vivo or in vitro method of claim 15 wherein a ligand binding to the antibody is indicative of a risk of developing an acute coronary syndrome (ACS) in a patient to whom the biological sample belongs.

18) The process of claim 2, wherein the polynucleotides are polynucleotide SEQ ID NOS:1 and 3 or any fragment thereof.
